(19) United States
(12) Patent Application Publication
(10) Pub. No.: US 2014/0235893 A1
(43) Pub. Date: Aug. 21, 2014

(54) Catalyst for Selectively Producing Acetic Acid Through the Partial Oxidation of Ethane

(71) Applicants: KING SAUD UNIVERSITY, Riyadh (SA); KING ABDULAZIZ CITY FOR SCIENCE AND TECHNOLOGY, Riyadh (SA)

(72) Inventors: Yousef S. Al-Zeghayer, Riyadh (SA); Moustafa A. Soliman, Riyadh (SA); Sulaiman Al-Mayman, Riyadh (SA); Abdulrhman S. Al-Awadi, Riyadh (SA)

(73) Assignees: KING ABDULAZIZ CITY FOR SCIENCE AND TECHNOLOGY, Riyadh (SA); KING SAUD UNIVERSITY, Riyadh (SA)

(21) Appl. No.: 13/772,736

(22) Filed: Feb. 21, 2013

(51) Int. Cl. B01J 27/199; C07C 51/215; B01J 23/652

(52) U.S. Cl. CPC B01J 27/199; B01J 23/6525; C07C 51/215
USPC 562/549; 502/312; 502/306; 502/309

(57) ABSTRACT

The invention relates to the development of a catalyst for selectively producing acetic acid from a gaseous feedstock of ethane, ethylene or mixtures thereof and oxygen at a low temperature. Said gaseous feedstock is brought together with a catalyst containing the oxides of Mo, V and Nb and nano metallic Pd optionally together with a hetero-poly acid and/or Sb and Ca. The present catalytic system provides both higher selectivity and yield of acetic with minimal production of side products of ethylene and CO.

### BACKGROUND OF THE INVENTION

1. Field of the Invention

The invention relates to improved catalysts, methods of manufacturing the same and methods of using the same in a process for low temperature oxydehydrogenation of ethane to acetic acid having reduced production of ethylene and CO by-products in the product stream based on the composition of the catalyst. The invention also relates to a one step vapor phase catalytic process using the novel catalyst featuring increased ethane conversion and higher selectivity to acetic acid up to 80% at particular process conditions.

2. Description of Related Art

Acetic acid is conventionally produced by methanol carbonylation using expensive rhodium catalysts in a liquid phase homogeneous reaction. This requires complicated procedures for recovery of the catalyst and isolation of products. More recently, acetic acid has been produced from an expensive raw material, ethylene, with the production of acetaldehyde as a by-product.

The use of molybdenum and vanadium-containing catalyst systems for low temperature oxydehydrogenation of ethane to ethylene was reported by E. M. Thorsteinson et. al., Journal of Catalysis, vol. 52, pp. 116-132 (1978). This paper discloses mixed oxide catalysts containing molybdenum and vanadium together with another transition metal oxide, such as Ti, Cr, Mn, Fe, Co, Ni, Nb, Ta, or Ce. The catalysts are active at temperatures as low as 200° C. for the oxydehydrogenation of ethane to ethylene. Some acetic acid is produced as a by-product at high pressure.

Several U.S. patents (U.S. Pat. Nos. 4,250,346, 4,524,236, 4,568,790, 4,596,787 and 4,899,003) have been granted on low temperature oxydehydrogenation of ethane to ethylene.

U.S. Pat. No. 4,250,346 discloses the use of a catalyst composition comprising the elements molybdenum, X and Y in a ratio of a:b:c for converting ethane into ethylene, where X is Cr, Mn, Nb, Ta, Ti, V and/or W and Y is Bi, Ce, Co, Cu, Fe, K, Mg, Ni, P, Pb, Sb, Si, Sn, TI and/or U and a is 1, b is from 0.05 to 1 and c is from 0 to 2. The total value of c for Co, Ni and/or Fe has to be less than 0.5. The reaction is preferably carried out in the presence of added water. The disclosed catalysts can likewise be used for the oxidation of ethane to acetic acid, with the efficiency of the conversion to acetic acid being about 18% at an ethane conversion of 7.5%.

U.S. Pat. No. 4,524,236 discloses catalysts useful for the production of ethylene from ethane via oxidative dehydrogenation, including oxides of molybdenum: MoₐV_{b}Nb_{c}Sb_{d}Xₑ, where X═Li, Sc, Na, Be, Mg, Ca, Sr, Ba, Ti, Zr, Hf, Y, Ta, Cr, Fe, Co, Ni, Ce, La, Zn, Cd, Hg, Al, Tl, Pb, As, Bi, Te, U, and W. The reaction can be carried out in the presence or absence of water; however, significant amounts of acetic acid are formed in the presence of water, which results in reduced ethylene selectivity.

The above cited patents are concerned mainly with the preparation of ethylene from ethane by the oxydehydrogenation process, less with the targeted preparation of acetic acid.

European Patent Publication EP 02 94 845 discloses a process for the higher selective production of acetic acid by the oxidation of ethane with oxygen in contact with a physical mixture of catalysts consisting of (A) a catalyst for oxydehydrogenation of ethane to ethylene and (B) a catalyst for hydration/oxidation of ethylene. The ethane oxydehydrogenation catalyst is represented by the formula MoₐV_{b}Z_{c}, wherein Z can be one or more of the metals Nb, Sb, Ta, Ca, Sr, Ti and W. The catalyst for hydration/oxidation is selected from a molecular sieve catalyst, a palladium-containing oxide catalyst, tungsten-phosphorus oxides, or a tin molybdenum containing oxide catalysts. When using the catalyst mixture described and passing a gas mixture comprising ethane, oxygen, nitrogen and water vapor through the reactor containing the catalyst, the maximum selectivity is 27% at an ethane conversion of 7%. This patent does not disclose the catalyst of the present invention which is designed in such a way that it has both oxydehydrogenation and oxygenation properties on the same catalyst.

Showa Denko [EP 0 62 0205 A1] relates to a catalytic process for converting ethylene to acetic acid using catalysts containing heteropoly acids of phosphorus, silicon, boron, aluminum, germanium, titanium, zirconium, cerium, cobalt, chromium and metal palladium with at least one element selected from groups XI, XIV, XV, and XVI of the periodic table. The single pass conversion of ethylene was reported to be very low over these heteropoly catalysts and produces a significant amount of acetaldehyde, which can have a great impact on the separation cost. The catalytic systems used in the present invention are different from the Showa Denko catalysts.

The selective preparation of acetic acid by catalytic gas-phase oxidation of ethane and/or ethylene in the presence of a palladium-containing catalyst is described in three patents by Karim et al. (U.S. Pat. Nos. 6,030,920, 6,310,241, 6,383,977) which have been granted to SABIC as a result of work carried out at King Saud University. In these patents, it is shown that the addition of Pd to the MoVNb catalyst greatly increases the selectivity to acetic acid to about 80% and completely oxidizes CO to CO₂.

The use of a palladium-containing catalyst is also described in U.S. Pat. Nos. 6,194,610, & 6,399,816. A gaseous feed comprising ethane, ethylene, and oxygen are brought into contact with a catalyst comprising the elements Mo, Pd, X, and Y in the gram-atomic ratios a:b:c:d in combination with oxygen: MoₐPd_{b}X_{c}Y_{d}. X represents one or more of Cr, Mn, Ta, Ti, V, Te, and W. Y represents one or more of B, Al, Ga, In, Pt, Zn, Cd, Bi, Ce, Co, Rh, Ir, Cu, Ag, Au, Fe, Ru, Os, K, Rb, Cs, Mg, Ca, Sr, Ba, Nb, Zr, Hf, Ni, P, Pb, Sb, Si, Sn, Ti and U. The indices a, b, c, and d represent the gram-atomic ratios of the corresponding elements: a=1, b=0.0001 to 0.01, c=0.4 to 1, and d=0.005 to 1.

The catalysts described in the abovementioned application (U.S. Pat. No. 6,194,610) gives a maximum space-time yield of 149 kg/hm3 at an acetic acid selectivity of >60 mol %. The best catalyst has the composition of Mo₁₆V₄Nb_{1.92}Pd_{0.008}. Space-time yields characterize the amount of acetic acid produced per unit time and catalyst volume. (U.S. Pat. No. 6,399,816). reported space-time yield of 470 kg/hm³ at an acetic acid selectivity of >80 mol % for a catalyst of composition Mo₁₆V_{8.8}Nb_{1.44}Pd_{0.0012}Ca_{0.28}Sb_{0.28}. Higher space-time yields are desirable since the size of the reactors and the amount of circulated gas can be reduced thereby.

There remains a need to develop a catalyst for the oxidation of ethane and/or ethylene to acetic acid and a process for the production of acetic acid using said catalyst and wherein the catalyst enables a high selectivity to acetic acid to be achieved.

### SUMMARY OF THE INVENTION

Surprisingly, it has now been found that by using a catalyst comprising molybdenum, vanadium, niobium and nano-metallic palladium in combination with oxygen, and wherein the molybdenum, vanadium, niobium and nano-palladium are present in specific amounts, ethane and/or ethylene may be oxidized to acetic acid with increased selectivity to acetic acid. Furthermore, it has been found possible using the catalysts of the present invention, to achieve a high selectivity to acetic acid with reduced selectivity to ethylene. In addition the amount of metallic palladium used is at least one third of the amount of palladium in the oxidized form. This is a great saving in the amount of expensive palladium.

Accordingly, the present invention provides a catalyst composition for the oxidation of ethane and/or ethylene to acetic acid, which composition comprises the elements molybdenum, vanadium and niobium in combination with oxygen and palladium that is introduced in nano-metallic form.

An advantage of catalyst compositions according to the present invention is that they can be more active and selective in converting ethane and/or ethylene to acetic acid than compositions not according to the present invention. Another advantage of the present catalyst composition is that the amount of palladium used is less than one third of the amount of palladium that is used in previous art. The selectivity to acetic acid that may be achieved is greater than 70%, such as greater than 80%. In particular, using the catalyst compositions of the present invention, a high selectivity to acetic acid with carbon dioxide as side product and excellent space time yield, may be achieved in combination with a low, if any, selectivity to ethylene and carbon monoxide.

It is therefore an object of the invention to provide a process which allows ethane and/or ethylene to be oxidized to acetic acid in a simple and targeted manner and at high selectivity and space-time yield under reaction conditions which are as mild as possible.

According to an aspect of the present invention, a catalyst may include oxides of Mo, V and Nb and nano metallic Pd, the catalyst having a composition represented by the formula MoₐV_{b}Nb_{c}Pd_{d}Oₓ wherein a is 16, b is 4 to 10, c is 0.2 to 4, and d is 0.001 to 0.02, and wherein x is a number determined by the valence requirements of the other elements in the catalyst composition.

The catalyst may further include a hetero-poly acid and/or Sb and Ca.

The catalyst may be a supported catalyst further including a support.

The support may include a microporous material, a nanoporous material or mixtures thereof.

The supported catalyst may include from 10 to 50% by weight catalyst composition and 50 to 90% by weight support.

The catalyst may be a supported catalyst further comprising a titania support.

According to another aspect of the present invention, a method of manufacturing the catalyst including the steps of: a) forming a mixture comprising Mo, V, Nb and Pd in a solution; b) drying said mixture to form a dried solid material; and c) calcining said dried solid material to form said catalyst.

The mixture may be formed by combining a first solution with a second solution, wherein said first solution and said second solution each contain at least one element selected from the group consisting of Mo, V, Nb and Pd.

The calcining may include heating said dried solid material to a calcining temperature from about 250° C. to about 450° C. in air or oxygen for a period of time ranging from about one hour to about 16 hours.

According to another aspect of the present invention, a process of producing acetic acid from a gaseous feed including ethane plus oxygen, or a mixture of ethane and ethylene plus oxygen, at an elevated temperature, the process including bringing the gaseous feed into contact with the catalyst.

The reaction temperature may be in the range from 200 to 400° C.

The process may occur in a reactor, wherein a pressure in the reactor is in the range from 10 to 50 bar.

Ethane mixed with at least a further gas may be fed to a reactor in which the process occurs. The further gas may be at least one of nitrogen, oxygen, carbon monoxide, carbon dioxide, ethylene or water vapor.

The catalyst may include at least one of the following compositions in combination with oxygen: a) Mo₁₆V_{6.37}Nb_{2.05} Pd_{0.0037}; and b) Mo₁₆V_{6.37}Nb_{2.05} Pd_{0.0037}Sb_{0.28} Ca_{0.28}.

The catalyst may be mixed with a support material or fixed on a support material.

The selectivity of the oxidation reaction of ethane and/or ethylene to acetic acid may be >70 mol %.

According to another aspect of the present invention, there is a catalyst for selective oxidation of ethane to acetic acid, the catalyst made by a process including the steps of: a) combining the elements Mo, V, Nb and Pd in the following ratio to form a composition having the formula: MoₐV_{b}Nb_{c}Pd_{d}, wherein: a is 16; b is 4 to 10; c is 0.2 to 4; and d is 0.001 to 0.02; and b) calcining said composition to form said catalyst.

The catalyst may be a supported catalyst further comprising a support, such as a titania support.

### DESCRIPTION OF THE PREFERRED EMBODIMENT(S)

According to the present invention, ethane is oxidized with molecular oxygen to acetic acid in a gas phase reaction at relatively high levels of conversion, selectivity and productivity and at temperatures ranging from 150° C. to 450° C. and at pressures ranging from 1-50 bar. This is achieved using a catalyst having a calcined composition of MoₐV_{b}Nb_{c}PdₐOₓ, wherein: a is 16; b is 4 to 10; c is 0.2 to 4; and d is 0.001 to 0.02.

The numerical values of a, b, c and d represent the relative gram-atom ratios of the elements Mo, V, Nb and Pd, respectively, in the catalyst. The elements Mo, V and Nb are present in combination with oxygen in the form of various oxides. The inventive catalysts are preferably produced using the methods disclosed herein.

The catalyst is a mixture of the elements in combination with oxygen. The catalyst can also be represented by the formula Mo₁₆V₄₋₁₀ Nb₀₂₋₄ Pd_{0.001-0.02}Oₓ where x is a number determined by the valence requirements of the other elements in the catalyst composition.

The catalyst of the invention can be used with or without a support. Preferred support materials may be a microporous material, a nanoporous material or mixtures thereof, and preferably have a surface area of less than 100 m²/g. Suitable supports for the catalyst include alumina, silica, titania, zirconia, zeolites and mixtures thereof. Preferably the support is titania (P25) from Degussa. When used on a support, the supported catalyst usually comprises from about 10 to 50% by weight of the catalyst composition, with the remainder being the support material.

The catalyst can be used as a heterogeneous oxidation catalyst after shaping as regularly or irregularly shaped support bodies or else in powder form.

Preferably, the catalyst is prepared from a solution of water-soluble substances such as ammonium salts, nitrates, sulfates, halides, hydroxides and salts of organic acids of each of the metals which can be converted into the corresponding oxides by heating. To mix the components, aqueous solutions or suspensions of the metal salts are prepared and mixed so as to provide a desired gram-atom ratios of the elements in the catalyst composition.

The solution is preferably an aqueous system having a pH of 1 to 10 and more preferably at a pH of 1 to 7, at a temperature of from about 30° C. to about 100° C. The catalyst composition is then prepared by removing the water and/or other solvent from the mixture of the compounds in the solution system. The dried catalyst is calcined by heating to a temperature from about 250° C. to about 450° C. in air or oxygen for a period of time from about one hour to about 16 hours to produce the desired catalyst composition. Generally, the higher the temperature the shorter the period of time required.

Preferably, the molybdenum is introduced into the solution in the form of ammonium salts such as ammonium paramolybdate, or organic acid salts of molybdenum such as acetates, oxalates, mandelates, and glycolates. Some other partially water soluble molybdenum compounds which may be used include molybdenum oxides, molybdic acid, and chlorides of molybdenum.

Preferably, the vanadium is introduced into the solution in the form of ammonium salts such as ammonium metavanadate and ammonium decavanadate, or organic salts of vanadium such as acetates, oxalates, and tartrates. Partially water soluble vanadium compounds such as vanadium oxides, and sulfates of vanadium can be used. To achieve a complete solubility, a certain amount of oxalic or tartaric acid can be added. The niobium is preferably used in the form of oxalates. Other sources of this metal in soluble form include compounds in which the metal is coordinated, bonded or complexed to a beta-diketonate, carboxylic acid, and amine, and alcohol, or an alkanolamine.

Preferably palladium is introduced as nano metallic using any method known in the literature such as that described by Y. Z. Chengcai Luo, and Yanguang Wang, Journal of Molecular Catalysis A: Chemical, vol. 229, pp. 7-12, 2005 for preparation of metallic nano-palladium by exploiting polyethylene glycol(PEG) which was found to act as both reducing agent and stabilizer. The preparation of nano-palladium was very straightforward. Palladium acetate (25 mg, 110×10⁻³ mmol) was added into PEG(molecular weight 4600, 2.0 g, 1.0 mmol) in a 50 mL round-bottomed flask under vigorous stirring by magnetic stirring at 120° C. The resulting homogenous solution was maintained at 120° C. with further stirring for 2.0 h. This resulted in conversion of transparent solution to the characteristic gray black color of nano Pd. The mixture solidifies when cooling to room temperature. The size of the nano-Pd depends among other things on the molecular weight of PEG.

Any other palladium compounds can be used, for example, palladium(II) chloride, palladium(II) sulfate, tetramminepalladium(II) nitrate, palladium(II) nitrate and palladium(II) acetylacetonate.

Palladium contents in the catalyst which are above the upper limit indicated promote carbon dioxide formation in the process of the invention. Furthermore, higher palladium contents are generally also avoided because they make the catalyst unnecessarily expensive. On the other hand, palladium contents below the limit indicated favor formation of ethylene.

The gaseous mixture is generally introduced into a reaction zone having a temperature of about 150 to about 400 or 450° C., and preferably 170 to 350° C., more preferably 200 to 300° C. The reaction zone generally has a pressure of 1 to 50 bar, and preferably 1 to 30 bar, the contact time between the reaction mixture and the catalyst is about 0.01 to 100 seconds, and preferably 0.1 to 10 seconds, and the space hourly velocity is about 50 to about 50,000 h-1, and preferably about 500 to 20,000 h-1 and more preferably from 100 to 10,000 h-1. The process is generally carried out in a single stage fixed bed or fluidized bed reactor, or solid moving bed reactor wherein all the oxygen and reactants are supplied as a single feed, with non-reacted starting materials being recycled. However, multiple stage addition of oxygen to the reactor with intermediate hydrocarbon feed can be used. This may improve productivity and avoid potentially hazardous conditions such as hydrocarbon and oxidant explosive mixture or mixture of hydrocarbon and oxidant in explosive envelope, generation of hot spots which ultimately affect the product distribution and catalyst life.

Preferably, the catalyst is prepared by the following general procedure. Aqueous solution of vanadium, niobium and molybdenum are prepared separately. The vanadium solution is mixed with molybdenum solution at particular temperature and pH. Palladium is slowly added to the combined gel solution. The niobium solution is added to the MoVPd solution to form a combined gel. After mixing and heating for about ½ to 2 hours, the resultant gel is dried to incipient wetness with continuous stirring at about 100° C. The resultant gel mixture is dried at 120° C. for 16 hours and then it is heated to 350° C. at the rate of 1° per minute and calcined at this temperature in air for half an hour to produce the desired oxide composition. This regime seems to be close to optimum as it allows to obtain a catalyst with the desired structure.

Another aspect of the invention relates to the production of acetic acid from ethane without the production or with significantly reduced production of the by-products ethylene and CO in the product stream.

According to one embodiment of the invention, acetic acid is produced directly from ethane on a single step vapor phase catalytic process using the catalyst according to the invention.

The reaction mixture useful in carrying out the process is generally one mole of ethane, 0.01 to 0.5 moles of molecular oxygen or higher by applying of proper measures to avoid explosion problems either as pure oxygen or in the form of air, and zero to 4.0 moles of water in the form of steam. The water vapor or steam is used as a reaction diluent and as a heat moderator for the reaction and it also increases the selectivity to acetic acid. Other gases may be used as reaction diluents or heat moderators such as helium, nitrogen, and carbon dioxide.

The gaseous components of the reaction mixture include ethane, oxygen and a diluent, and these components are uniformly admixed prior to being introduced into the reaction zone. The components may be preheated, individually or after being admixed, prior to being introduced into the reaction zone.

The gaseous feed comprises ethane and/or ethylene which are fed to the reactor as pure gases or in admixture with one or more other gases. Examples of such additional or carrier gases are nitrogen, methane, carbon monoxide, carbon dioxide, air and/or water vapor. The gas comprising molecular oxygen can be air or a gas comprising more or less molecular oxygen than air, e.g. oxygen. The proportion of water vapor can be in the range from 0 to 50% by volume. Higher water vapor concentrations would make the work-up of the aqueous acetic acid formed unnecessarily expensive for process reasons. The ratio of ethane/ethylene to oxygen is advantageously in the range from 1:1 to 10:1, preferably from 2:1 to 8:1. Relatively high oxygen contents are preferred since the achievable ethane conversion and thus the yield of acetic acid is higher. Oxygen or the gas comprising molecular oxygen is preferably added in a concentration range outside the explosive limits under the reaction conditions since this makes the process easier to carry out. However, it is also possible to employ an ethane/ethylene to oxygen ratio within the explosive limits.

According to one preferred embodiment, the process is carried out in a single stage with all the oxygen and reactants being supplied as a single feed with unreacted initial reactants being recycled. However, multiple stage addition of oxygen to the reactor with an intermediate hydrocarbon feed can also be used. This may improve productivity to acetic acid and avoid a potentially hazardous condition.

The mixed gases are preferably preheated to the reaction temperature in a preheating zone before the gas mixture is brought into contact with the catalyst. The reaction is carried out at temperatures of from 150 to 350° C., preferably from 200 to 300° C. The pressure can be atmospheric or superatmospheric, e.g. in the range from 1 to 50 bar, preferably from 1 to 30 bar. A contact time between the reaction mixture and the catalyst of from about 0.01 second to 100 seconds, preferably from 0.1 second to 10 seconds; and a space hourly velocity of from about 50 to about 50,000 h-1, preferably from 100 to 10,000 h-1 and most preferably from 200 to 3,000 h-1.

The contact time is defined as the ratio between the apparent volume of the catalyst bed and the volume of the gaseous reaction mixture feed to the catalyst bed under the given reaction conditions in a unit of time.

The space velocity is calculated by determining total reactor outlet gas equivalent in liters of the total effluent evolved over a period of one hour divided by the liters of catalyst in the reactor. This room temperature volume is converted to the volume at 0° C. at 1 bar.

The reaction can be carried out in a fixed-bed or fluidized-bed reactor. Acetic acid is separated from the gas leaving the reactor by condensation. The remaining gases are recirculated to the reactor inlet where oxygen or the gas comprising molecular oxygen and also ethane and/or ethylene are metered in. The catalyst of the invention is not limited to the oxydehydrogenation of ethane to ethylene and acetic acid and may be used for oxidation of ethylene to acetic acid. The following examples are intended to be illustrative of this invention. They are, of course, not to be taken to in any way to limit the scope of this invention. Numerous changes and modifications can be made with respect to the invention without departing from the spirit or scope of the present invention.

On comparing the catalysts of the invention with those of the prior art, it is found that the present catalysts achieve higher space-time yields and acetic acid selectivities under identical reaction conditions (reaction feed gas, pressure, temperature). When using the catalyst of the invention, the selectivity in the oxidation of ethane and/or ethylene to acetic acid is >70 mol %, preferably >80 mol %, and the space-time yield is >500 g acetic acid/h.kg catalyst so that the process of the invention enables, in comparison with the prior art, an increase in the acetic acid yields to be achieved in a simple manner while simultaneously reducing the formation of undesired by-products.

### Catalysts Preparation

The catalyst composition described in the examples is given in relative atom ratios. For comparison, catalysts having a composition similar to the catalysts that are mentioned in U.S. Pat. No. 6,194,610 and U.S. Pat. No. 6,399,816 were prepared.

### Comparative Catalyst (A)

A catalyst having a composition similar to the U.S. Pat. No. 6,194,610 catalyst was prepared. The catalyst is not according to the present invention because it does not contain nano metallic palladium. The catalyst composition was:

Mo₁₆V_{8.8}Nb_{1.44}Pd_{0.012}

Ammonium metavanadate in the amount 0.794 grams was dissolved in 35 milliliters distillated water and heated to 70° C. with stirring for 15 minutes to obtain a clear yellow color solution (solution A). 0.69 grams of niobium oxalate containing 21.5% by weight calculated as Nb₂O₅ were added to 30 milliliters of water and heated to 65° C. with white color solution (solution B). Solution B was then added to solution A. The combined mixture was stirred at 70° C. for 15 minutes (mixture C). Ammonium para molybdate tetrahydrate in the amount of 2.16 grams was added to 20 milliliters of distillated water and heated to 60° C. with stirring to give colorless solution (solution D). Mixture C and solution D were combined together and stirred at 70° C. for 15 minutes to get mixture E.2.0×10⁻² grams of palladium acetate was dissolved in 5.0 milliliters acetone and stirred for 10 minutes. Palladium solution was finally added to the mixture E with stirring at 70° C. for 15 minutes. This combination was stirred vigorously to achieve a homogenous gel mixture which was the dried slowly to incipient dryness within 120-180 minutes at 85-90° C. The resulting solid was put in the desiccator to the next day and then dried further in a furnace at 120° C. for sixteen hours. After that furnace temperature was raised from 120 to 350° C. at rate 1.0° C./minutes and thereafter held at 350° C. for a half hour. Calcined catalyst was cooled to room temperature and formulated into uniform particles.

### Comparative Catalyst (B)

A catalyst having a composition similar to the U.S. Pat. No. 6,399,816 catalyst was prepared. Catalyst is not according to the present invention because it does not contain nano metallic palladium. The catalyst composition was:

Mo₁₆V_{8.8}Nb_{1.44}Pd_{0.012}Sb_{0.28}Ca_{0.28}.

The procedures and amounts of the components were similar to what was carried out in Comparative Catalyst (A) except that antimony (III) chloride (0.049 grams) and calcium nitrate (0.035 grams) were added to niobium solution (solution B).

The following examples are illustrative of some of the products and methods of making the same falling within the scope of the present invention. They contain nano metallic palladium.

The following examples are according to the invention. An important step in the preparation method for supported catalyst is to add the niobium salt solution after the addition of titania to the other salts solution as suggested by Xu & Iglesia, Applied Catalysis A: General, vol. 334, pp. 339-347, 2008.

### EXAMPLE 1

A catalyst was prepared to have the following composition:

Mo₁₆V_{6.37}Nb_{2.05}Pd_{0.0037}Sb_{0.28}Ca_{0.28}.

Ammonium metavanadate in the amount 0.57 grams was dissolved in 25 milliliters distillated water and heated to 85° C. with stirring for 15 minutes. 1.50 grams of oxalic acid were added to metavanadate clear yellow color solution to obtain a dark blue solution (solution A). Ammonium para molybdate tetrahydrate in the amount of 2.16 grams was added to 20 milliliters of distillated water and heated to 60° C. with stirring to give colorless solution (solution B). Solution B was then combined with solution A. The combination was heated at 87° C. with continuous stirring to give solution C. The solution color change from dark blue to dark green olives.

2 grams of poly ethylene glycol (Molecular weight 4600) was put in 60 ml flask, malted by heating at 40° C. for 7 minutes. The stirrer temperature was raised to 120° C. at rate 5° C./minute and thereafter held at 120° C. for 10 minute. 25.0 milligrams of palladium acetate were added very slowly with vigorous stirring to achieve to a homogeneous gel mixture. The brown color mixture was changed to gray, dark gray and finally black color.

This resulted in conversion of transparent solution to the characteristic of palladium nano-particles. The mixture was stirred vigorously for 2.0 hours at 120° C. and then stirrer temperature was decreased to 75° C. Distillated water was added slowly with stirring to get a homogenous solution with 50 milliliter total volume.

The required amount of metallic nano-palladium was collected and dropped slowly to mixture C (mixture formed by mixing ammonium metavanadate and ammonium paramolybdate solutions). 0.97 grams of niobium oxalate containing 21.5% by weight calculated as Nb₂O₅ were added to 30 milliliters of water and heated to 65° C. with white color solution. Antimony (III) chloride (0.049 grams) and calcium nitrate (0.035 grams) were added to niobium solution. The combination was heated at 65° C. with continuous stirring to give mixture (D). Mixture (D) was dropped very slowly to mixture C with continuous stirring at 85° C. This combination was stirred vigorously to achieve a homogenous gel mixture which was then dried slowly to incipient dryness within 120-180 minutes at 85-90° C. with continuous stirring. The resulting solid was put in the desiccator to the next day and then dried further in a furnace at 120° C. for sixteen hours. After that furnace temperature was raised to 350° C. at rate 1.0° C./minute and thereafter held at 350° C. for a half hour. Calcined catalyst was cooled to room temperature and formulated into uniform particles.

### EXAMPLE 2

Catalyst having the following composition was prepared:

Mo₁₆V_{6.37}Nb_{2.05}Pd_{0.0055}Sb_{0.28}Ca_{0.28}

The procedure and amounts of the components were similar to what was carried out in example 1 except that 50% more of metallic nano palladium was used.

### EXAMPLE 3

For comparison, the catalyst having a composition similar to the catalyst in example 2 but without Antimony and calcium was prepared and tested. The composition of this catalyst is:

Mo₁₆V_{6.37}Nb_{2.05}Pd_{0.0055}

The catalyst was prepared in accordance with the procedure used in example 1 except that 50% more of metallic nano palladium was used and Antimony and calcium were not included.

### EXAMPLE 4

A catalyst having the same composition as the catalyst in example 2 was supported on titania P25. Thus the catalyst composition is:

Mo₁₆V_{6.37}Nb_{2.05}Pd_{0.0055}Sb_{0.28}Ca_{0.28}/TiO₂

The catalyst was prepared using the same procedures of examples 1 and 2 except that the Titania in the amount 5.0 grams was impregnated very slowly by the combination solution formed by mixing ammonium metavanadate solution (A), ammonium para molybdate tetrahydrate solution (B) and the required amount of metallic nano-palladium with continuous stirring at 85° C. to give dark green to dark gray color mixture. Niobium oxalate solution included both antimony chloride and calcium nitrate then was dropped slowly to the above mixture with continuous stirring at 85° C. This combination was stirred vigorously to achieve a homogenous gel mixture which was the dried slowly to incipient dryness within 120-180 minutes at 85-90° C. the drying and calcination procedures were the same as used in Example 1. Catalyst loading is 30%.

### EXAMPLE 5

The catalyst of example 4 was prepared except that antimony and calcium were not included and the amount of metallic nano palladium was the same as the catalyst in example 1 the composition of this catalyst was:

Mo₁₆V_{6.37}Nb_{2.05} Pd_{0.0037}/TiO₂

Catalyst loading is 30%.

### EXAMPLE 6

Catalyst of example 5 was repeated except that phosphotungstic acid was added. Phosphotungstic containing catalyst was prepared by adding 0.2 grams of phosphotungstic acid to the combination solution formed by mixing ammonium metavanadate solution (A), ammonium para molybdate tetrahydrate solution (B) and the required amount of metallic nano-palladium with continuous stirring at 85° C. Drying and calcination of catalyst were done by following the procedure mentioned in Example 1. Catalyst loading is 30%.

### Method of Catalyst Testing

Catalysts evaluations were carried out in a stainless steel straight tube reactor heated in an oven and having an internal diameter of 4.00 millimeters under standard process conditions. The gas feed composition was 82% by volume ethane and 18% by volume oxygen at a pressure of 15 bar at a flow rate of about 15 milliliters/minute and space velocity at about (3000 h-1) by using of 0.3 grams of calcined catalyst. In case of supported catalyst, the reactor contained 1.0 grains of calcined catalyst; the reactor bed depth was about 10 centimeters, so that the depth to cross-section ratio was about 25. The reaction temperature was measured by means of a thermocouple in the catalyst bed. Reaction products were analyzed on-line by gas chromatography. Columns packed with material sold under the trademarks PORAPAK-QC and CARBOXEN 1000 were used for products analysis.

In all case, the conversion and selectivity calculation were based on the stoichiometry:

C₂H₆+0.5 O₂→C₂H₄+H₂O

C₂H₆+1.5 O₂→CH₃COOH+H₂O

C₂H₆+2.5 O₂→²CO+3H₂O

C₂H₆+3.5 O₂→2CO₂+3H₂O

In the examples, the following terms are defined as:

Ethane conversion (%)=100*([CO]/2+[CO2]/2+[C2H4]+[CH3COOH])/([CO]/2+[CO2]/2+[C2H2]+[C2H6]+[CH3COOH]) where [ ]=concentrations in mol % and [C2H6]=concentration of the unreacted ethane.

The yield of acetic acid was calculated by multiplying the selectivity to acetic acid by ethane conversion.

The space time yield (STY) of acetic acid was calculated by multiplying the feed flow rate by the yield of acetic acid divided by the catalyst weight.

Since the space-time yield is dependent on the reaction pressure, all comparative examples were carried out at 15 bar for reasons of comparability.

Total conversion of ethane decreases with the increase in the selectivity of acetic acid. Selectivity to acetic acid passes through a maximum with an increase in the amount of palladium in the mixed oxide catalysts. Further, the amount of ethylene and carbon monoxide (primary reaction products) can be completely converted to acetic acid and carbon dioxide, depending on the composition of the catalyst.

Reaction conditions and results are summarized in the table 1.

The following observation can be made from table 1:

1. STY of Example 2, 4 and 6 of the present invention are better than previous art in this field of catalysts A and B.

2. The addition of antimony and calcium reduces selectivity of COₓ.

3. The addition of hetero-poly acid, such as phosphotungstic acid, also reduces selectivity of COx.

4. Supporting the catalyst on titania improve ethane conversion and selectivity of acetic acid.

We obtained STY of about 600g acetic acid/h.kg of catalyst for catalyst 6 whereas comparative catalyst B gave about 430 g acetic acid/h.kg of catalyst at higher temperature.

By reducing the contact time we can increase the temperature and thus we can obtain higher STY of acetic acid as shown in table 2 where we reached about 2000 g acetic acid/h.kg of catalyst never reached before in any previous art in this filed.

TABLE 1
Catalyst testing result
(Feed flow rate is 12.3 C₂H₆ and 2.70 O₂ ml/min,
pressure is 15 bar and catalyst weight is 0.3 g for
unsupported catalyst and 1.0 g for supported catalyst)

| Ex- | | Oxy- | Eth- | Selectivity (%) | | | STY g/kg · h | |
|---|---|---|---|---|---|---|---|---|
| am- | | gen | ane | | Ace- | | | Ace- |
| ple | Temp | Con. | Con. | Ethyl- | tic | | Ethyl- | tic |
| NO. | C. | % | % | ene | Acid | COₓ | ene | Acid |
| A | 250 | 47.6 | 5.53 | 0 | 83.58 | 16.42 | 0 | 304.55 |
| | 275 | 94.84 | 7.77 | 0 | 70.78 | 29.22 | 0 | 362.38 |
| B | 250 | 22.04 | 2.41 | 0 | 86.24 | 13.76 | 0 | 136.95 |
| | 275 | 71.45 | 8.03 | 0 | 81.09 | 18.91 | 0 | 429.05 |
| 1 | 225 | 20.18 | 2.33 | 29.01 | 62.69 | 8.29 | 20.79 | 96.25 |
| | 240 | 37.69 | 3.39 | 18.85 | 67.08 | 14.07 | 19.65 | 149.86 |
| 2 | 250 | 36.36 | 4.44 | 22.73 | 65.24 | 12.02 | 31.03 | 190.86 |
| | 275 | 88.13 | 9.63 | 9.84 | 71.83 | 18.33 | 29.14 | 455.78 |
| 3 | 250 | 55.25 | 8.03 | 52.18 | 38.6 | 9.22 | 204.24 | 128.84 |
| | 260 | 88.12 | 11.26 | 43.49 | 44.53 | 11.98 | 330.38 | 150.58 |
| 4 | 220 | 35.73 | 4.73 | 2.77 | 84.25 | 12.98 | 4.03 | 262.58 |
| | 240 | 77.07 | 8.21 | 1.24 | 81.11 | 17.66 | 3.13 | 438.77 |
| 5 | 240 | 99.63 | 10.21 | 0.66 | 83.21 | 16.13 | 2.07 | 559.79 |
| | 250 | 100 | 10.25 | 0.78 | 82.71 | 16.51 | 2.46 | 558.61 |
| 6 | 240 | 89.68 | 10.66 | 0.82 | 85.85 | 13.35 | 2.69 | 603.01 |
| | 250 | 99.88 | 10.15 | 0.618 | 81.81 | 17.12 | 1.92 | 547.14 |

TABLE 2
Testing results of catalyst example 5
(Feed composition is 18% O₂ and 82% C₂H₆,
reaction pressure is 15 bar and catalyst weight is 0.2 g)

| | | Oxy- | Eth- | Selectivity (%) | | | STY g/kg · h | |
|---|---|---|---|---|---|---|---|---|
| Flow | | gen | ane | | Ace- | | | Ace- |
| rate | Temp | Con. | Con. | Ethyl- | tic | | Ethyl- | tic |
| Ml/min | C. | % | % | ene | Acid | COₓ | ene | Acid |
| 45 | 275 | 18.09 | 2.09 | 19.04 | 67.15 | 13.81 | 183.5 | 1387.11 |
| 30 | 275 | 27.9 | 3.45 | 11.61 | 71.82 | 16.55 | 123.17 | 1632.64 |
| 15 | 275 | 61.21 | 7.27 | 2.01 | 81.71 | 16.27 | 22.47 | 1957.07 |
| 9 | 275 | 96.68 | 9.98 | 1.3 | 82.03 | 16.67 | 11.97 | 1618.27 |

The above description of the invention is intended to be illustrative and not limiting. Various changes or modifications in the embodiments described may occur to those skilled in the art. These can be made without departing from the spirit or scope of the invention.

The invention claimed is:

1. A catalyst comprising oxides of Mo, V and Nb and nano metallic Pd, the catalyst having a composition represented by the formula MoₐV_{b}Nb_{c}Pd_{d}Oₓ wherein
a is 16,
b is 4 to 10,
c is 0.2 to 4, and
d is 0.001 to 0.02, and
wherein x is a number determined by the valence requirements of the other elements in the catalyst composition.

2. The catalyst of claim 1, further comprising a hetero-poly acid and/or Sb and Ca.

3. The catalyst of claim 1, wherein said catalyst is a supported catalyst further comprising a support.

4. The catalyst of claim 3, wherein said support comprises a microporous material, a nanoporous material or mixtures thereof.

5. The catalyst of claim 3, wherein said supported catalyst comprises from 10 to 50% by weight catalyst composition and 50 to 90% by weight support.

6. The catalyst of claim 1, wherein said catalyst is a supported catalyst further comprising a titania support.

7. A method of manufacturing the catalyst of claim 1, comprising the steps of:
a) forming a mixture comprising Mo, V, Nb and Pd in a solution;
b) drying said mixture to form a dried solid material; and
c) calcining said dried solid material to form said catalyst.

8. The method of claim 7, wherein said mixture is formed by combining a first solution with a second solution, wherein said first solution and said second solution each contain at least one element selected from the group consisting of Mo, V, Nb and Pd.

9. The method of claim 7, wherein said calcining comprises heating said dried solid material to a calcining temperature from about 250° C. to about 450° C. in air or oxygen for a period of time ranging from about one hour to about 16 hours.

10. A process of producing acetic acid from a gaseous feed comprising ethane plus oxygen, or a mixture of ethane and ethylene plus oxygen, at an elevated temperature, the process comprising bringing the gaseous feed into contact with the catalyst of claim 1.

11. The process as claimed in claim 10, wherein a reaction temperature is in the range from 200 to 400° C.

12. The process as claimed in claim 10, wherein the process occurs in a reactor, wherein a pressure in the reactor is in the range from 1 to 50 bar.

13. The process as claimed in claim 10, wherein ethane mixed with at least a further gas is fed to a reactor in which the process occurs.

14. The process as claimed in claim 13, wherein the further gas is at least one of nitrogen, oxygen, carbon monoxide, carbon dioxide, ethylene or water vapor.

15. The process as claimed in claims 10, wherein the catalyst comprises at least one of the following compositions in combination with oxygen:
a) Mo₁₆V_{6.37}Nb_{2.05}Pd_{0.0037}; and
b) Mo₁₆V₆₃₇Nb_{2.05}Pd_{0.0037}Sb_{0.28} Ca_{0.28}

16. The process as claimed in claim 10, wherein the catalyst is mixed with a support material or fixed on a support material.

17. The process as claimed in claim 10, wherein the selectivity of the oxidation reaction of ethane and/or ethylene to acetic acid is >70 mol %.

18. A catalyst for selective oxidation of ethane to acetic acid, the catalyst made by a process comprising the steps of:
a) combining the elements Mo, V, Nb and Pd in the following ratio to form a composition having the formula:
MOₐV_{b}Nb_{c}Pd_{d}
a is 16;
b is 4 to 10;
c is 0.2 to 4; and
d is 0.001 to 0.02; and
b) calcining said composition to form said catalyst.

19. The catalyst of claim 18, wherein said catalyst is a supported catalyst further comprising a support.

20. The catalyst of claim 18, wherein said catalyst is a supported catalyst further comprising a titania support.
